(19) United States
(12) Patent Application Publication
(10) Pub. No.: US 2019/0314680 A1
(43) Pub. Date: Oct. 17, 2019

(54) NOISELESS EXERCISE SYSTEM USING EMG

(71) Applicant: Roemsystem Corp., Daejeon (KR)

(72) Inventors: Jung Jin Hwang, Sejong-si (KR); Young Dae Seo, Sejong-si (KR)

(21) Appl. No.: 16/132,700

(22) Filed: Sep. 17, 2018

(30) Foreign Application Priority Data
Apr. 11, 2018 (KR) 10-2018-0042307

(51) Int. Cl. A63B 24/00; A61B 5/0488; A61B 5/11; A63B 21/002; A61B 5/22; A63B 71/06; A63B 21/00; A63B 23/035; A63B 23/04; A63B 23/12

(52) U.S. Cl. CPC A63B 24/0062; A63B 2071/0658; A61B 5/0488; A61B 5/1116; A63B 21/0023; A61B 5/224; A63B 71/0622; A63B 21/00072; A63B 23/03525; A63B 23/0405; A63B 23/1218; A63B 2230/08; A63B 2230/62; A63B 2225/10; A61B 2560/0475; A63B 2024/0065; A63B 2220/836; A63B 2225/09; A63B 2024/0071; A63B 2220/803; A63B 24/0075

(57) ABSTRACT

An exercise system comprises a stationary exercise apparatus including support arms, exercise parts, and a frame, at least one electromyograph configured to be worn on at least one body portion of a user, the electromyograph including an electromyogram sensor and a motion sensor, and a smart device configured to obtain a posture according to the detected motion, elect at least one exercise part corresponding to the posture from among the exercise parts, determine a target exercise part among the at least one exercise part elected, obtain a muscle strength by analyzing the detected electromyogram, record a muscle strength enhancement history in association with a use history of the determined target exercise part, and output the recorded muscle strength enhancement history and the use history.

### CROSS-REFERENCE TO RELATED APPLICATIONS

This patent application claims priority under 35 U.S.C. § 119 to Korean Patent Application No. 10-2018-0042307, filed on Apr. 11, 2018, in the Korean Intellectual Property Office, the disclosure of which is incorporated by reference herein in its entirety.

### TECHNICAL FIELD

Embodiments of the disclosure relate to fitness equipment, and more specifically, to noiseless exercise systems using electromyograms.

### DISCUSSION OF RELATED ART

Muscle contractions may be divided largely into isotonic and isometric contractions. Mostly, fitness equipment used in home or gym involve isotonic exercises. However, isotonic exercises may cause lots of noise for their structural reasons.

Isometric exercise or isometrics are a type of strength training in which the joint angle and muscle length do not change during contraction (compared to concentric or eccentric contractions, called dynamiclisotonic movements). Isometrics are done in static positions, rather than being dynamic through a range of motion.

In isometrics, it is not easy to assess muscle strength. Thus, isometric exercise equipment, to be able to assess muscle strength, may need sensors, thus causing the equipment complicated in structure.

Muscle strength may be assessed based on electromyogram.

Conventional exercise equipment require prior and individual entry of information for an exercise to be done and may show the user exercise strength, but not whether the user is properly doing exercise, whether electromyogram is detected from the muscle being used for exercise, what muscle is trained or strengthened, and whether the user is using the fitness equipment in an appropriate manner.

There may be known some fitness equipment that may trace the trajectory of the body portion during exercise to judge whether the exercise is being properly done. However, such equipment are inappropriate for isometric exercises.

### SUMMARY

According to embodiments of the disclosure, there are provided an EMG-based, noiseless exercise system that includes a noiseless stationary exercise apparatus for isometric exercises with a simplified structure for easier use, that may identify the kind of the isometric exercise which the user is currently doing to precisely assess the muscle(s) being used for the exercise.

According to an embodiment, an exercise system comprises a stationary exercise apparatus including a plurality of support arms each of which is provided per kind of exercise, a plurality of exercise parts installed on the support amis, and a frame to which the support arms are firmly fastened, at least one electromyograph configured to be worn on at least one body portion of a user, the electromyograph including an electromyogram sensor configured to detect an electromyogram from a muscle of the body portion and a motion sensor configured to detect a motion of the body portion, and a smart device configured to obtain a posture according to the detected motion, elect at least one exercise part corresponding to the obtained posture from among the plurality of exercise parts, determine a target exercise part, which is to be operated using a muscle from which an electromyogram is detected, among the at least one exercise part elected, obtain a muscle strength by analyzing the detected electromyogram, record a muscle strength enhancement history in association with a use history of the determined target exercise part, and output the recorded muscle strength enhancement history and the use history.

According to an embodiment, the motion sensor may be configured to detect a slope and the motion of the body portion, and wherein the smart device is configured to receive an input for the body portion from the user, upon detecting the motion that is not more than a threshold, detect the slope, and determine a posture corresponding to the detected slope based on a correlation between the slope of the body portion and a posture to be taken when the user does isometric exercise on the stationary exercise apparatus.

According to an embodiment, each of the at least one electromyographs may be worn on a respective corresponding one of the at least one body portion to individually detect an electromyogram from the corresponding body portion. According to an embodiment, at least one of the at least one electromyograph may be configured to individually detect an electromyogram from a plurality of muscles of a body portion corresponding thereto. According to an embodiment, the smart device may be configured to elect the at least one exercise part according to a plurality of muscles from which an electromyogram is detected.

According to an embodiment, the smart device may be configured to monitor the detected motion and the muscle from which the electromyogram is detected, when the posture is changed, re-elect at least one exercise part depending on the changed posture and the muscle from which the electromyogram is detected, and when the muscle from which the electromyogram is changed, re-elect the at least one exercise part depending on the muscle from which the electromyogram is detected from among the at least one exercise part elected according to the posture.

According to an embodiment, the smart device may be configured to convert the obtained muscle strength into a weight value and output the weight value on a screen.

According to an embodiment, the smart device may be configured to measure, via the electromyograph, an electromyogram detected when the user's weight applies in a posture of applying a force to one of the exercise part to correct a correlation between the muscle strength and the electromyogram so that the muscle strength obtained through the electromyogram is adjusted to the actual weight.

According to an embodiment, at least one of the plurality of exercise parts may be installed on a corresponding one of the support arms to have a gap in a direction along which a force is applied thereto, and the at least one exercise part is moved within a range of the gap when the force is applied and is returned to an original position by a returning means when the force is released.

According to an embodiment, the stationary exercise apparatus may include an angle adjuster configured to adjust an angle of each support arm or a length adjuster configured to adjust a length of each support arm.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a view illustrating an example of using an exercise system according to an embodiment;

FIG. 2 is a perspective view illustrating a stationary exercise apparatus according to an embodiment;

FIG. 3 is a block diagram illustrating an electromyograph and a smart device according to an embodiment;

FIG. 4 is a flowchart illustrating an exercise method performed under the control of a smart device according to an embodiment;

FIG. 5 is a front perspective view illustrating a stationary exercise apparatus in an exercise system according to an embodiment;

FIG. 6 is a rear perspective view illustrating a stationary exercise apparatus in an exercise system according to an embodiment;

FIG. 7 is a top perspective view illustrating a movement allowing structure for an exercise part; and

FIG. 8 is a perspective view illustrating a stationary exercise apparatus in a folded position according to an embodiment.

### DETAILED DESCRIPTION OF EXEMPLARY EMBODIMENTS

Hereinafter, exemplary embodiments of the disclosure will be described in detail with reference to the accompanying drawings. The same reference denotations may be used to refer to the same or similar elements throughout the specification and the drawings. The disclosure, however, may be modified in various different ways, and should not be construed as limited to the embodiments set forth herein. As used herein, the singular forms “a,” “an,” and “the” are intended to include the plural forms as well, unless the context clearly indicates otherwise.

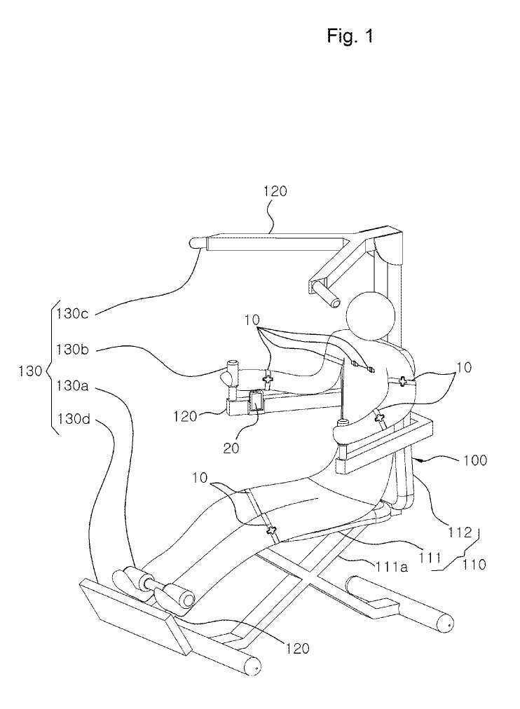

FIG. 1 is a view illustrating an example of using an exercise system according to an embodiment. Referring to FIG. 1, according to an embodiment, an exercise system includes a stationary exercise apparatus 100, an electromyographl0, and a smart device 20. The stationary exercise apparatus 100 remains stationary and is provided for muscle training or strengthening. The electromyograph 10 is worn on the user's body to detect the user's electromyogram and motion. The smart device 20 may identify the kind of the exercise using the electromyogram and motion and manage the history of enhancing muscle strength.

Unlike conventional stationary exercise equipment that, when a force is applied by muscular contraction, permit movement while receiving a resistance in the reverse direction of the force being applied, the stationary exercise apparatus 100 does not receive any resistance in the reverse direction and has little or no dynamic movement using joints. In other words, the stationary exercise apparatus 100 strengths the user's muscles by static exercises (i.e., isometric exercises).

The smart device 20 may grasp the status of the user's exercise using electromyograms and motions detected by the electromyograph 10 and analyze the user's exercise. Thus, even when the user frequently or randomly changes exercises, the smart device 20 may grasp and analyze the context on its own, thus eliminating the need for a separate sensor.

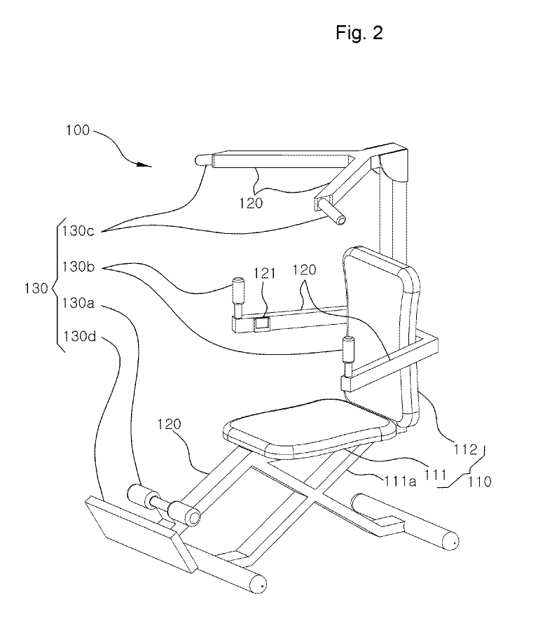

Embodiments of the disclosure are described below in greater detail with FIGS. 2 to 4.

FIG. 2 is a perspective view illustrating a stationary exercise apparatus 100 according to an embodiment.

The stationary exercise apparatus 100 includes a frame 110 that allows a user to stably sit, support arms 120 extending or protruding from the frame 110, and exercise parts 130 installed or provided on the support arms 120.

The frame 110 includes a leg 111a, a seat 111 placed on the leg 111a, and a backrest 112 with a proper height. During exercise, the user may sit on the seat 111 while leaning against the backrest 112. The shape or form of the frame 110 is not limited to the example shown in FIG. 2 and may rather be implemented in any other various configurations that allow the exercise parts 130 and the support arms 120 to be installed thereon.

The exercise parts 130 are parts of the stationary exercise apparatus 100, to which the user is to apply muscular forces for strengthening his muscles.

The support arms 120 are intended for providing the exercise parts 130 suited for the type of exercise that the user is to do. In conventional stationary exercise equipment, the support arms allow the exercise parts to move or reposition along the direction of the force applied thereto while receiving a resistance in the reverse direction.

However, in the stationary exercise apparatus 100 of the disclosure, although a force is applied to the exercise parts 130, the support arms 120 remain stationary, preventing the exercise parts 130 from repositioning or moving.

In other words, conventional stationary exercise equipment involve isotonic exercises or contractions whereas the stationary exercise apparatus 100 of the disclosure involves isometric exercises or contractions.

The isometric exercise using the stationary exercise apparatus 100, like isotonic exercise, repeats flexing and relaxing muscles, and hence, muscle contractions and relaxations, thereby strengthening the user's muscles.

The exercise parts 130 need to be placed at a proper height to allow a desired muscle to contract while the user sits on the seat. Considering this, the support arms 120 are installed on the frame 110.

A plurality of support arms 120 may be provided to strength a plurality of different muscles.

The exercise parts 130 may include an exercise part 130a for leg extension, an exercise part 130b for pec dec flies or butterflies, an exercise part 130c for pull-ups or shoulder press, and an exercise part 130d for the leg press. However, embodiments of the disclosure are not limited thereto, and more exercise parts 130 may be added for various types of exercises. Two exercise parts 130a and 130d may be installed on one support arm 120 as shown.

At least one of the support arms 120 may have a mount 121 for the smart device 20. The mount 121 may be provided in a position where the user can easily look and check. The smart device 20 may include a display 24. The user may look at the screen on the display 24. The mount 121 may also be used to store the electromyograph 10 when the stationary exercise apparatus 100 is not in use.

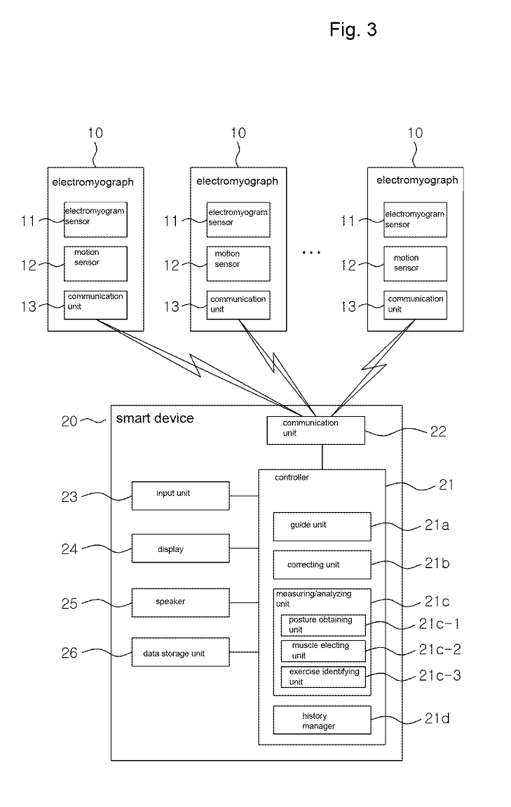

FIG. 3 is a block diagram illustrating an electromyograph 10 and a smart device 20 according to an embodiment.

The electromyograph 10 is configured to be worn on the user's body. The electromyograph 10 includes an electromyogram sensor 11 to detect electromyograms generated when the muscles in the body portion, where the electromyograph 10 is worn, contract, a motion sensor 12 to detect the slope of the electromyograph 10 and the movement or motion of the electromyograph 10, and a communication unit 13 to transmit detected electromyograms and motions to the smart device 20.

The electromyograph 10 may be configured in the form of a band or strap wearable on the user's body or a patch attachable onto the user's body. Alternatively, the electromyograph 10 may have a known structure to allow it to be worn on the user's body.

The motion sensor 12 of the electromyograph 10 may be, e.g., an accelerometer or acceleration sensor, a gyroscope sensor, a geo-magnetic sensor, an altitude sensor, or one of any other various sensors which are able to detect the motion of the user's body portion where the electromyograph 10 is worn.

According to an embodiment, there may be provided a plurality of electromyographs 10 that are worn on multiple body portions of the user.

Each electromyograph 10 may have a plurality of electromyogram sensors 11 to individually detect the electromyograms of multiple muscles of the user's body.

According to an embodiment, the plurality of electromyographs 10 may detect the respective electromyograms and motions of the body portions where the electromyographs 10 are worn, and one of the plurality of electromyographs 10 may gather or compile the electromyograms and motions from the electromyographs 10 and send the gathered or compiled electromyograms and motions to the smart device 20.

The smart device 20 includes a communication unit 22 to communicate with the electromyograph 10, an input unit 23 to receive user inputs, a display 24 to output various data, messages, images, or other various types of information on the screen, a speaker 25 to output sounds, a data storage unit 26 to store data, and a controller 21 to execute an application for performing steps, functions, or operations of an exercise method. The smart device 20 may be, e.g., a smart phone or tablet PC.

The communication unit 22 may be, or include, a short-range wireless communication module, such as a Bluetooth module, or a wired communication module for communication via a connector.

Data stored in the data storage unit 26 may include correlation information and exercise identification information stored when the application is installed.

The correlation information may be information that represents a correlation or relationship between the muscle strength and electromyogram generated when the muscle contracts.

An electromyogram signal may be analyzed in the time domain and the frequency domain by a known method to obtain various pieces of feature information. Such feature information may be used to obtain the muscle strength and the degree of muscle fatigue.

The data storage unit 26 may previously store a data table that represents a correlation between the information obtained from the electromyogram of the muscle corresponding to each exercise part 130 and the muscle strength generated as the muscle contracts, or the data storage unit 26 may previously store a program to obtain the muscle strength that may be obtained from the information obtained from the electromyogram.

According to an embodiment, the correlation between the electromyogram and the degree of muscle fatigue may be stored per muscle in the data storage unit 26. Hereinafter, the correlation between the electromyogram and the muscle strength may be referred to as a “muscle strength correlation” or “muscle strength correlation information,” and the correlation between the electromyogram and the degree of muscular fatigue may be referred to as a “muscle fatigue correlation” or “muscle fatigue correlation information.”

The muscle strength correlation information and the muscle fatigue correlation information may be obtained from an electromyogram that may be obtained when the user does isometric exercise using the stationary exercise apparatus 100.

The exercise identification information may include the posture of the user when the user applies a force to the exercise part 130 and the type or kind of the muscle that contracts when the force is applied to the exercise part 130. The exercise identification information may be divided per exercise part 130 of the stationary exercise apparatus 100.

Since the user may apply forces to different exercise parts 130 in similar postures or the user may apply a force to one of the exercise parts 130 to contract multiple muscles, postures and muscles each of which matches a respective one of the exercise parts 130 may overlappingly match different exercise parts 130 or some exercise part 130 may match a plurality of postures or a plurality of muscles. Such overlapping or multiple matching may cause it difficult to identify the exercise parts 130, but this issue may be addressed by the muscles from which postures and electromyograms are detected as set forth below.

The user's posture when he does exercise may be grasped by analyzing the user's motion, and exercise parts 130 which allows the user to take the posture may be narrowed down in kind. Although multiple exercise parts 130 are narrowed down, the target exercise part 130 which is applying the force can be identified using the muscle from which the electromyogram is detected.

The exercise identification information may be stored in the form of a data table that records the posture and muscle per exercise part 130 or in the form of a program able to narrow down exercise parts 130 which are operated per posture and then to determine the target exercise part 130 depending on the muscle from which the electromyogram is detected.

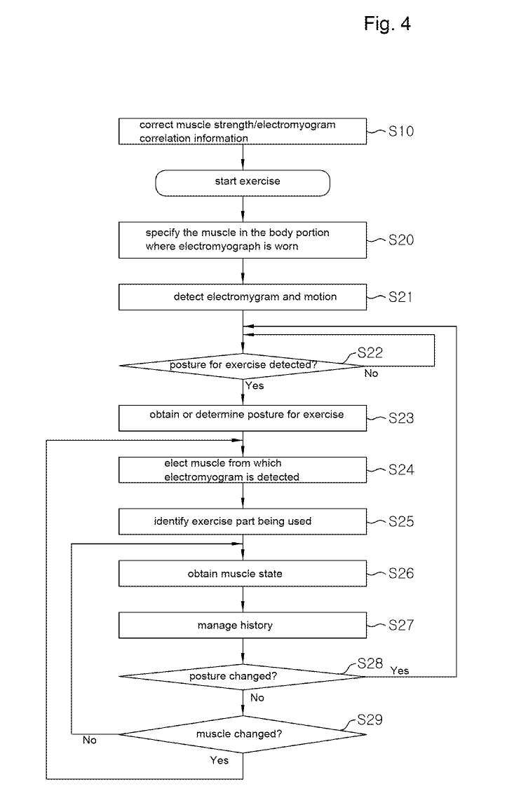

The application may implement or perform the functions of a guide unit 21a, a correcting unit 21b, a measuring/analyzing unit 21c, and a history manager 21d, and the application may execute the steps, functions, or operations of the exercise method as shown in FIG. 4. The application may be read by the smart device 20 and executed by the controller 21. When the application is read and executed by the smart device 20, the muscle strength correlation information, the muscle fatigue correlation information, and the exercise identification information may be stored in the data storage unit 26.

The guide unit 21a may output a screen through the display 24 or a sound through the speaker 25. Contents that are to be output by the guide unit 21 are generated when the correcting unit 21b and the measuring/analyzing unit 21c are operated.

The correcting unit 21b may correct the muscle strength correlation information to fit the user's muscle. For example, the correcting unit 21b may perform a correcting process for measuring, via the electromyograph 10, an electromyogram detected when the user's weight applies in the posture of applying a force to the exercise part 130, thereby correcting the correlation between the muscle strength and the electromyogram so that the muscle strength obtained through the electromyogram is adjusted to the actual weight.

The measuring/analyzing unit 21c may detect the electromyogram of the user who is doing exercise using the stationary exercise apparatus 100 to thereby assess the exercise. The measuring/analyzing unit 21c may detect the electromyogram of the muscle strength corresponding to the exercise part and the posture identified using the motion and the electromyogram and assess the exercise.

For example, the measuring/analyzing unit 21c may specify, by a user input or a worn portion notification, the body portion where the electromyograph 10 is worn and the muscle from which the electromyogram is measured or detected by the electromyograph 10.

The measuring/analyzing unit 21c includes a posture obtaining unit 21c-1 to obtain a posture according to the motion of the body portion where the electromyograph 10 is worn, a muscle electing unit 21c-2 to elect the muscle from which the electromyogram is detected, and an exercise identifying unit 21c-3 to identify, or elect, the target exercise part 130, which is to be operated using the muscle from which the electromyogram is detected, among the exercise parts 130 narrowed down. The measuring/analyzing unit 21c determines the target exercise part 130.

The measuring/analyzing unit 21c analyzes the electromyogram of the muscle corresponding to the determined target exercise part 130, thereby obtaining the muscle strength.

The target exercise part 130 may precisely be determined in terms that the user does isometric exercise using the stationary exercise apparatus 100. The measuring/analyzing unit 21c, upon detecting that the detected motion is a preset threshold or less, may detect the slope of the body portion where the electromyograph 10 is worn and select the posture corresponding to the detected slope based on the correlation between the slope and the posture that the user takes upon doing isometric exercise on the stationary exercise apparatus 100.

The measuring/analyzing unit 21c may obtain the degree of muscle fatigue from the electromyogram, analyze the waveform of the electromyogram to count the number of times in which the force is applied to the exercise part 130, and determine the exercise strength according to the counted number of times.

The history manager 21d may, in real-time, receive the kind of the exercise part 130, the kind of exercise, muscle strength, exercise strength, and degree of muscle strength from the measuring/analyzing unit 21c, record and manage the history, and provide an exercise guide screen to output the real-time received information and a history managing screen to output a use history for each exercise part 130 and a history of the muscle strength enhanced by each exercise part 130.

The functions or operations of the guide unit 21a, the correcting unit 21b, the measuring/analyzing unit 21c, and the history manager 21d are embodied in the flowchart of FIG. 4 illustrating an exercise method.

FIG. 4 is a flowchart illustrating an exercise method performed by the controller 21 executing the application, according to an embodiment.

Referring to FIG. 4, the exercise method includes a correcting step S10 which is performed by the correcting unit 21b before the exercise starts.

In the correcting step S10, the electromyograph 10 is worn on the user's muscle corresponding to the exercise part 130, and the user is then guided to do muscle strength strengthening exercise, by which the actual weight is applied, in the same posture as applies a force to the exercise part 130. The actual weight applied is guided to input to the input unit 23. The muscle strength correlation information about the muscle corresponding to the exercise part 130 is corrected based on the electromyogram detected by the electromyograph 10 as the exercise proceeds.

In other words, since the same type of stationary exercise apparatus as the stationary exercise apparatus 100 may be equipped in a gym, the user may do exercise on the stationary exercise apparatus. Since a force is applied to the weight stack of the stationary exercise apparatus in the reverse direction of the exercise, the actual muscle strength correlation information about the user's muscle can be obtained by obtaining the electromyogram detected upon exercise and the weight of the weight stack. The weight of the weight stack is entered by the user.

The muscle strength correlation information stored in the data storage unit 26 may be corrected based on the so-obtained actual muscle strength correlation information. For example, although the integrated electromyogram (IEMG) or root mean square (RMS) used to assess muscle strength may differ per user even when the same force is applied, the correlation with the muscle strength may be maintained. Thus, despite correcting the muscle strength correlation information with the IEMG or RMS obtained when the user does exercise one time while resisting a particular weight plate, muscle strength correlation information which reflects the correlation between the muscle strength and the electromyogram may be obtained on the electromyogram that may be shown to be different as the muscle strength varies.

Since the correcting process is performed to obtain the muscle strength correlation information for isometric exercise, the exercise in the correcting process is preferably isometric exercise.

The muscle strength correlation information correcting step S10 may preferably be performed on each of the plurality of exercise parts 130, but without being limited thereto, may be performed only on some exercise parts 130.

For the muscles corresponding to the exercise parts 130 for which no correction has been made, non-corrected muscle strength correlation information may be used. In this case, upon exercise, the user may adopt, as critical information, the strengthened degree of muscle which is a result of relative analysis based on the correlation between muscle strength and electromyogram and adopt the muscle strength as reference information.

When the user does exercise using the stationary exercise apparatus 100, the measuring/analyzing unit 21c and the history manager 21d are operated.

A muscle specifying step S20 is performed to specify the muscle in the body portion where the electromyograph 10 is worn.

Specifying the muscle may be carried out in a scheme in which the user himself enters, to the input unit 23, the body portion where the electromyograph 10 is worn or a scheme in which the body portion where the electromyograph 10 is worn is notified through the speaker 25 or the display 24.

Once the body portion where the electromyograph 10 is worn is specified, the muscle from which an electromyogram may be measured or detected may be specified based on the position of the electromyogram sensor 11 of the electromyograph 10 and the muscular distribution of the user's body.

As a plurality of electromyographs 10 may be worn or an electromyograph 10 capable of measuring or detecting a plurality of muscles may be worn, a plurality of muscles may be specified.

A detecting step S21 is performed to receive, via the communication unit 22, the electromyogram and motion detected by the electromyogram sensor 11 and the motion sensor 12 of the electromyograph 10.

A waiting step S22 is then performed to analyze the detected motion and to wait until the user takes a proper posture to the stationary exercise apparatus 100.

For example, information about the posture in which isometric exercise is performed while applying a force to the exercise part 130 of the stationary exercise apparatus 100 is previously stored. The slope is detected by the motion sensor 12 of the electromyograph 10 worn on the body portion, and the process waits until the slope becomes the one when the posture is taken.

A condition requiring that the motion be a preset threshold or less may be added. A motion may be detected by the motion sensor 12, and under the condition, the process may wait until the slope condition and the motion condition both are met.

In other words, since the user takes a nearly motionless posture upon exercising on the stationary exercise apparatus 100 although a slight motion occurs when he applies a force to the exercise part 130, a threshold may previously be set given the slight motion. When a threshold or less level of motion is detected with the slope condition met, the user's posture is determined to take a posture for the exercise.

In addition to the slope condition and the motion condition, another condition may be imposed requiring that an electromyogram by muscle contraction be detected by the electromyogram sensor 11. For example, such a condition that the power of an electromyogram signal corresponding to muscle contraction be detected may be added.

Since a plurality of postures may be taken depending on the kind of the exercise part 130 upon exercising on the stationary exercise apparatus 100, the process waits until any one of the plurality of postures is detected.

When a posture for exercise is detected in the waiting step S22, a posture obtaining step S23 is performed to determine the detected posture.

Here, the posture may be determined to be a posture having a correlation with the slope of the body portion where the electromyograph 10 is worn. However, there may be a posture difficult to determine with the slope alone. Thus, the posture may be determined by analyzing both the slope and the motion. In other words, since the body portion with the muscle to exert a force to the exercise part 130 presents a particular trajectory of motion per exercise part 10 and takes the posture, the trajectory may be used to determine the posture.

A muscle electing step S24 is performed to elect the muscle from which an electromyogram due to muscle contraction is detected. In other words, in the muscle electing step S24, only muscles where muscle contraction occurs are elected except for muscles where no muscle contraction occurs, among a plurality of muscles specified based on the body portion where the electromyograph 10 is worn.

Then, an exercise part identifying step S25 is carried out to select, based on the exercise identification information, the exercise part 130 that meets both the posture obtained in the posture obtaining step S23 and the muscle elected in the muscle electing step S24.

For example, exercise parts 130 corresponding to the posture are elected, and the exercise part 130 which is operated by the muscle from which an electromyogram is detected is elected among the elected exercise parts 130.

Since there may be a plurality of muscles where an electromyogram may be detected, it is possible to precisely identify any one exercise part 130 related to the plurality of muscles where an electromyogram is detected as the one being currently used for the user's exercise.

Referring back to FIGS. 1 and 2, the exercise part 130b for pec dec fly or butterfly and the exercise part 130c for pull up or shoulder up press may be distinguished from each other by the posture obtained using the motion sensor 12 of the electromyograph 10 worn on the user's arm.

However, the exercise part 130a for leg extension and the exercise part 130d for leg press might be difficult to distinguish only with the posture obtained using the motion sensor 12 of the electromyograph 10 worn on the leg. In such case, since different muscles are used for the exercise parts, the exercise parts may be distinguished from each other using the detected motion and the muscles being used. In this case, the electromyograph 10 needs to be equipped with a plurality of electromyogram sensors 11 along the circumstance of the leg.

According to an embodiment, although various types of exercise parts 130 are used, the exercise part 130 which matches both the posture and the muscles being used may be identified based on the exercise identification information.

After the muscles corresponding to the identified exercise part 130 may be narrowed down based on the exercise identification information, a muscle state obtaining step S26 is performed to obtain the muscle strength, degree of muscle fatigue, and exercise strength by analyzing the electromyogram detected from the muscles narrowed down among the plurality of muscles specified.

The muscle strength and degree of muscle fatigue are obtained based on the muscle fatigue correlation information and muscle strength correlation information about the muscles narrowed down.

The exercise strength is obtained by analyzing the temporal variation pattern of the electromyogram and the number of repetitions of muscle contraction.

Typically, the muscle strength is enforced by repeating several sets of muscle contraction and muscle relaxation. Since the magnitude (or power) of an electromyogram signal is larger upon muscle contraction than upon muscle relaxation, the number of exercise sets and the muscle contraction count per set may be obtained by the variation in the magnitude of the electromyogram signal.

Periods when the muscle contracts are extracted from the electromyogram signal and are analyzed in the time domain and the frequency domain, and the muscle strength which is based on the muscle strength correlation information and the degree of muscle fatigue which is based on the muscle fatigue correlation information are obtained per period.

The obtained muscle strength may be the tension generated by the muscle contraction and may correspond to the weight of the weight stack in the stationary exercise apparatus. Since the muscle strength is typically assessed with the weight of the weight stack, the obtained muscle strength may be converted into a weight value.

The muscle strength and degree of muscle fatigue which vary as the user does more sets of exercise are obtained with the muscle strength and degree of muscle fatigue distinguished per set.

The history manager 21d may perform a history management step S27 in which the history manager 21d may, in real-time, receive the results of assessment including the posture, exercise part, degree of muscle fatigue, and exercise strength obtained by the measuring/analyzing unit 21c and store the results of assessment in the data storage unit 26, may manaue the use history for the exercise part including the exercise strength, the enhancement history for the muscle strength per muscle, and the history for the muscle fatigue degree varying pattern during exercise, may provide a real-time exercise guide screen to output the results of assessment in real-time, and may provide a history management screen to output the history for the muscle strength enhanced by each exercise part 130 and the use history for each exercise part 130.

The muscle state obtaining step S26 and the history management step S27 may be repeated during the exercise.

The muscle strength and the degree of muscle fatigue vary as the muscle contraction count increases or as the number of sets increases and are recovered a sufficient time after the exercise is finished, and the muscle mass increases. The muscle strength and the degree of muscle fatigue are also varied depending on muscle mass.

The variation in the muscle strength and the variation in the degree of muscle fatigue during exercise may precisely be analyzed by differentiating the muscle strength and the degree of muscle fatigue obtained based on the electromyogram per muscle contraction period and per set. When the degree of muscle fatigue exceeds a predetermined threshold, the user may be notified of this so that the user may stop exercise.

The assessment about the muscle corrected as set forth above represents the absolute muscle strength for the user's muscle and is thus reliable. However, the assessment about the muscle strengthened without correction does not represent the absolute muscle strength for the user's muscle and may thus be less reliable. Thus, the user needs to take, as critical information, a relative assessment that represents the degree of enhancement whenever he does exercise.

For the muscle strength and the degree of muscle fatigue which vary as the number of sets increases, the muscle may be assessed with, e.g., differences between the sets and varying speed, and the results of the assessment may be compared with the results of assessment for the prior exercise and the user may be notified of the results of the comparison.

The measuring/analyzing unit 21c monitors, in real-time, the posture, and when the posture changes, performs the waiting step S22 (step S28), thus obtaining the exercise part, muscle strength, degree of muscle fatigue, and exercise strength for the changed posture.

The measuring/analyzing unit 21c monitors, in real-time, the muscle from which an electromyogram is detected during the exercise, and if the muscle where an electromyogram is detected is changed without a change in the posture in step S29, the measuring/analyzing unit 21c performs the muscle electing step S24.

In other words, the muscle state is obtained fitting the muscle and exercise part corresponding to the changed posture and the history is managed. The exercise part used may be varied if the muscle used is varied in the same posture. In this case, thus, identifying the exercise part is re-performed, and the muscle state is obtained and the history is managed.

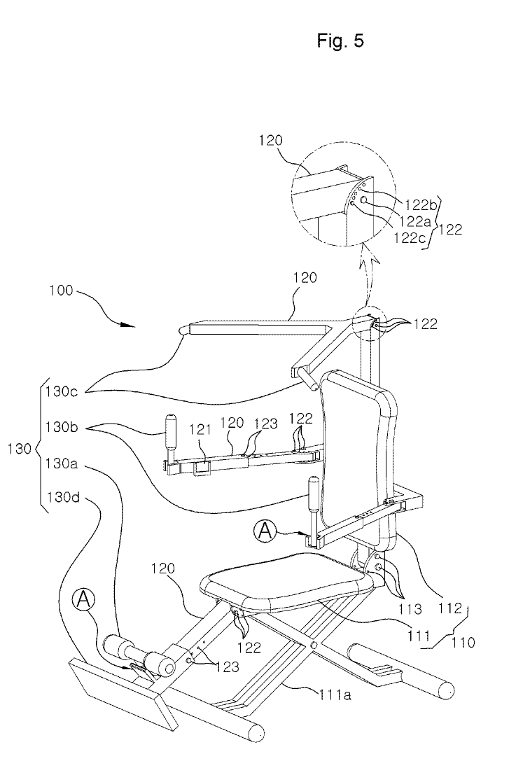

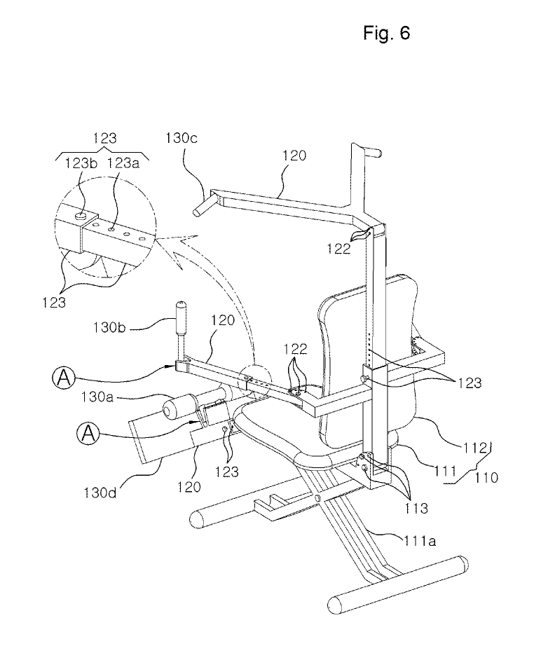

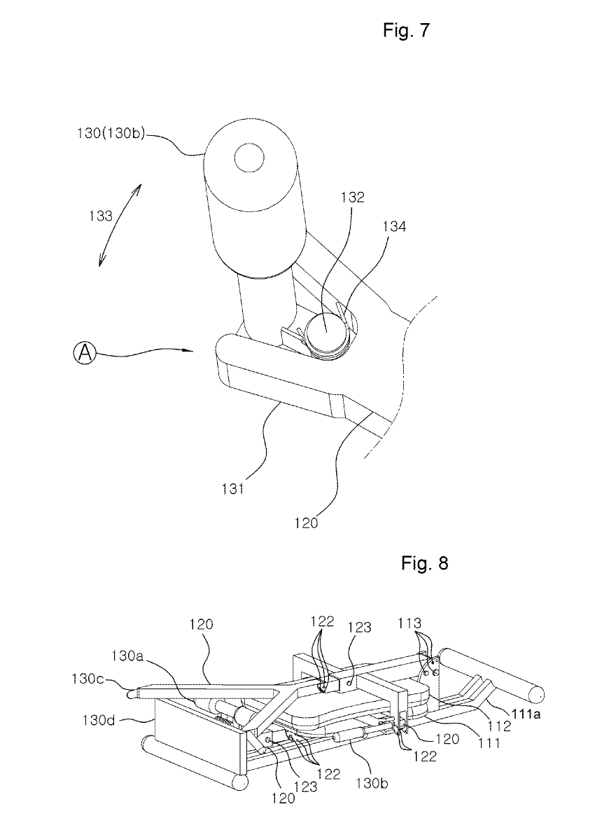

FIGS. 5 to 8 are views illustrating a stationary exercise apparatus 100 according to an embodiment. FIG. 5 is a front perspective view illustrating the stationary exercise apparatus 100, FIG. 6 is a rear perspective view illustrating the stationary exercise apparatus 100, FIG. 7 is a top perspective view illustrating a movement allowing structure A for the exercise part 130, and FIG. 8 is a perspective view illustrating the stationary exercise apparatus 100 in a folded position.

According to an embodiment, at least any one of a plurality of support arms 120 provided in the stationary exercise apparatus 100 includes an angle adjuster 122 to adjust the angle by rotation or a length adjuster 123 to adjust the length by extension or contraction.

At least one 130a and 130b of exercise parts 130 installed on their respective corresponding support arms 20 has a movement allowing structure A that permits movement when a force is applied thereto and returns to the original position when the force is released.

In this embodiment, unlike the above-described embodiment in which the support arms 120 are motionlessly fastened to the frame 110, the angle adjuster 122 is provided in the position where the support arm 120 is fixed or in the middle of the support arm 120 so that the support arm 120, after turning at a desired angle, can be fixed motionless.

Referring to FIG. 5, specifically, the enlarged part of FIG. 5, two arm parts are separately produced and then combined together, forming the support arm 120 that is bendable in the middle thereof. In other words, a first one of the arm parts has an exercise part 130 at one end thereof, and the other arm part is rotatably combined with the first arm part. The first arm part is angled about the other arm part, and in this position, an adjusting pin 122c passes through the two arm parts to maintain the angle. A plurality of through holes 122b may be formed through the arm parts so that the first arm part can be adjusted at various angles. The angle adjuster 122 includes the adjusting pin 122c and a plurality of through holes 122b.

Upon forming an angle adjuster 122 in the support arm 120 with an exercise part 130a for leg extension and an exercise part 130d for leg press, the angle adjuster 122 may be formed in an end of the support arm 120 fixed to the frame 110 so that the overall support arm 120 may be angle-adjusted.

The angle adjuster 122 is not limited to those shown in the drawings and may be formed in other various structures that allow the exercise part 130 installed on the support arm 120 to be repositioned by rotation.

The support arm 120 may be angle-adjusted and then be fixed to be stopped from movement. The exercise part 130 may be redirected with respect to a rotating shaft 122a, allowing for a diversity of isometric exercise and allowing different muscles to be strengthened with one exercise part 130.

For example, the angle of the exercise part 130a for leg extension may be adjusted by the angle adjuster 122 formed in the support arm 120 so that the user may change the angle of the legs. The exercise part 130a for leg extension may be adjusted to the height of the set 111 of the frame 110 so that the user may do seated leg curls in an isometric exercise manner.

The angle adjuster 122 may turn the support arm 120 to be placed tightly close to the frame 110, thus allowing the stationary exercise apparatus 100 to be put in a folded position when not in use.

The stationary exercise apparatus 100 may include a folding means 113 that allows the backrest 112 to be folded and placed tightly close to the seat 111, allowing the stationary exercise apparatus 100 to be stored in a folded position.

The length adjuster 123 may adjust the length of the support arm 120 to change the position of the exercise part 130 installed on the support arm 120. Thus, the position of the exercise part 130 may be adjusted to fit the user's body.

Referring to the enlarged part of FIG. 6, the length adjuster 123 allows the two arm parts of the support arm 120, which are shaped as, e.g., square bars, to be coupled together in such a manner that one of the arm parts is end-to-end inserted into the other arm part. Holes 123a are formed in an end of one of the arm parts, and an adjusting pin 123b, which passes through an end of the other arm part, is inserted into one of the holes 123a.

A length adjuster 123 may be formed in an end, fastened to the frame 110, of the support arm 120 with an exercise part 130c for pull ups installed thereon. In other words, the length adjuster 123 may be formed in the middle of the support arm 120 or in an end thereof which is fastened to the frame 110.

However, the length adjuster 123 is not limited to the structure shown in the drawings, and a conventional arm length adjusting structure may be adopted for the length adjuster 123.

The length adjuster 123 allows the support arm 120 to be placed in a minimized length when the stationary exercise apparatus 100 is in a folded position, thus allowing for easy and convenient storage or carrying of the stationary exercise apparatus 100.

The angle adjuster 122 or the length adjuster 123 may be formed in some or all of the plurality of support arms 120 with the exercise parts 130.

The movement allowing structure A of the exercise part 130 is described below with reference to FIG. 7.

The exercise part 130 is installed on the support arm 120 to have a gap 133 to permit a slight movement in the direction along which the force is applied. As a force is applied to the exercise part 130, the exercise part 130 may be moved in the gap, and when the force is released, the exercise part 130 is returned to the prior position by a returning means 134.

Referring to FIG. 7, the movement allowing structure A formed in the exercise part 130b for pec dec fly includes a rotating shaft 132 provided to rotate the exercise part 130 in a direction along which a force is applied, a fixing member 131 having the rotating shaft 132 installed therein, fastened to the support arm 120, and having wings to restrict the range of rotation of the exercise part 130 and a returning means 134 configured with a torsion spring to return the exercise part 130, which has been moved in the gap within which movement is permissible, to the oritinal position.

Referring to FIG. 6, a movement allowing structure A is also formed in the exercise part 130a for leg extension and is operated in substantially the same manner as the movement allowing structure A formed in the exercise part 130b.

A movement allowing structure A may also be formed in the exercise part 130c for pull ups or the exercise part 130d for leg press.

Although the movement allowing structure A of the exercise part 130 is formed in a rotatable structure, the movement allowing structure A may be formed in a structure that permits movement back and force. For example, a movement allowing structure A that permits back-and-force movement may preferably be formed in the exercise part 130d for leg press.

The movement allowing structure A permitting back-and-force movement may require no rotating shaft 132, fasten the exercise part 130 to a fixing member to be moveable back and forth within the range of the gap, and allow the exercise part 130 to be returned to the original position by a returning means configured with a contraction or expansion spring. When a force is applied to the exercise part 130 in an advancing direction, the exercise part advances within the range of the gap, and when the force is released, the exercise part 130 moves back to the original position.

Although in the above example, the movement allowing structure A is formed in a portion where the exercise part 130 is coupled with the support arm 120, embodiments of the disclosure are not limited thereto. The movement allowing structure A may be formed in the middle of the support arm 120 or in a portion where the support arm 120 and the frame 110 are coupled together. Such variations differ from the above-described embodiment only in terms of the position where the movement allowing structure A is formed, and no detailed description thereof is thus given.

According to an embodiment, the movement allowing structure A may be formed in the angle adjuster 122 or the length adjuster 123. For example, the hole through which the adjusting pin 122c passes in the angle adjuster 122 may be formed to be elongate along the direction in which the force is applied, thus allowing the adjusting pin 122c to move within the range of the hole while making an elastic restoring force applied to the support arm 120 in a direction opposite to the direction in which the force is applied. The movement allowing structure A may be formed in the length adjuster 123 in substantially the same manner as the angle adjuster 122.

FIG. 8 illustrates the stationary exercise apparatus 100 in a folded position. The support arms 120 are positioned tightly close to the frame 110 by the angle adjusters 122 while being rendered as short as possible by the length adjusters 123, the seat 111 and the backrest 112 are folded by the folding means 113, and the legs 111a are folded. Any known structure used in typical fitness equipment may be adopted to fold the legs 111a.

The stationary exercise apparatus 100 may easily be stored and carried when it is in the folded position.

As set forth above, the stationary exercise apparatus 100 does not require a weight stack and a resisting means constituted of tension cables or other cables to deliver the weight of the weight stack nor does it need any sensing means, and may thus be made lighter, simpler, and more compact. Further, the stationary exercise apparatus 100 creates no noise, thus allowing the user to enjoy exercise without being bothered with noise. This makes the stationary exercise apparatus 100 appropriate for home use.

The stationary exercise apparatus 100 includes the angle adjuster 122, the length adjuster, and the movement allowing structure A, thereby allowing for various kinds of isometric exercise and easier storage and carrying.

Although the user randomly changes the kinds of exercise, the stationary exercise apparatus 100 may identify the kind of exercise that the user is currently doing and assess the exercise.

According to the embodiments of the disclosure, the exercise system adopts the stationary exercise apparatus including exercise parts configured not to move, preventing noise while the user does exercise.

Although the user randomly or frequently changes the kind of isometric exercise, the exercise system may identify the exercise that the user is currently doing and exactly spot and analyze the muscle(s) associated with the identified exercise, thus able to present precise analysis results for various kinds of exercises.

The exercise parts may be permitted a slight movement, thus allowing the user to enjoy isometric exercises with more fun.

Further, the exercise system may convert a muscle strength into a weight value and show the weight value, thus putting the user on notice about what strength the user is doing the exercise in.

The exercise system may correct errors in muscle strength that may occur differences from person to person and present absolute muscle strengths.

The angle adjuster or the length adjuster of the exercise system allows for diversified kinds of exercises and easier carrying and storage.

While the disclosure has been shown and described with reference to exemplary embodiments thereof, it will be apparent to those of ordinary skill in the art that various changes in form and detail may be made thereto without departing from the spirit and scope of the disclosure as defined by the following claims.

What is claimed is:

1. An exercise system, comprising:
a stationary exercise apparatus including a plurality of support arms each of which is provided per kind of exercise, a plurality of exercise parts installed on the support arms, and a frame to which the support arms are firmly fastened;
at least one electromyograph configured to be worn on at least one body portion of a user, the electromyograph including an electromyogram sensor configured to detect an electromyogram from a muscle of the body portion and a motion sensor configured to detect a motion of the body portion; and
a smart device configured to obtain a posture according to the detected motion, elect at least one exercise part corresponding to the obtained posture from among the plurality of exercise parts, determine a target exercise part, which is to be operated using a muscle from which an electromyogram is detected, among the at least one exercise part elected, obtain a muscle strength by analyzing the detected electromyogram, record a muscle strength enhancement history in association with a use history of the determined target exercise part, and output the recorded muscle strength enhancement history and the use history.

2. The exercise system of claim 1, wherein the motion sensor is configured to detect a slope and the motion of the body portion, and wherein the smart device is configured to receive an input for the body portion from the user, upon detecting the motion that is not more than a threshold, detect the slope, and determine a posture corresponding to the detected slope based on a correlation between the slope of the body portion and a posture to be taken when the user does isometric exercise on the stationary exercise apparatus.

3. The exercise system of claim 2, wherein each of the at least one electromyographs is worn on a respective corresponding one of the at least one body portion to individually detect an electromyogram from the corresponding body portion, wherein at least one of the at least one electromyograph is configured to individually detect an electromyogram from a plurality of muscles of a body portion corresponding thereto, and wherein the smart device is configured to elect the at least one exercise part according to a plurality of muscles from which an electromyogram is detected.

4. The exercise system of claim 1, wherein the smart device is configured to monitor the detected motion and the muscle from which the electromyogram is detected, when the posture is changed, re-elect at least one exercise part depending on the changed posture and the muscle from which the electromyogram is detected, and when the muscle from which the electromyogram is changed, re-elect the at least one exercise part depending on the muscle from which the electromyogram is detected from among the at least one exercise part elected according to the posture.

5. The exercise system of claim 1, wherein the smart device is configured to convert the obtained muscle strength into a weight value and output the weight value on a screen.

6. The exercise system of claim 1, wherein the smart device is configured to measure, via the electromyograph, an electromyogram detected when the user's weight applies in a posture of applying a force to one of the exercise part to correct a correlation between the muscle strength and the electromyogram so that the muscle strength obtained through the electromyogram is adjusted to the actual weight.

7. The exercise system of claim 1, wherein at least one of the plurality of exercise parts is installed on a corresponding one of the support arms to have a gap in a direction along which a force is applied thereto, and the at least one exercise part is moved within a range of the gap when the force is applied and is returned to an original position by a returning means when the force is released.

8. The exercise system of claim 1, wherein the stationary exercise apparatus includes an angle adjuster configured to adjust an angle of each support arm or a length adjuster configured to adjust a length of each support arm.
